(19) United States
(12) Patent Application Publication
(10) Pub. No.: US 2021/0246474 A1
(43) Pub. Date: Aug. 12, 2021

(54) METHOD FOR CONTINUOUS SECOND-GENERATION ETHANOL PRODUCTION IN SIMULTANEOUS SACCHARIFICATION AND CO-FERMENTATION PROCESS

(71) Applicants: Indian Oil Corporation Limited, Mumbai (IN); Department Of Biotechnology, New Delhi (IN)

(72) Inventors: Ajay Kumar SHARMA, Faridabad (IN); Manas Ranjan SWAIN, Faridabad (IN); Ajit SINGH, Faridabad (IN); Anshu Shankar MATHUR, Faridabad (IN); Ravi Prakash GUPTA, Faridabad (IN); Deepak TULI, Faridabad (IN); Suresh Kumar PURI, Faridabad (IN); Sankara Sri Venkata RAMAKUMAR, Faridabad (IN)

(21) Appl. No.: 17/212,528

(22) Filed: Mar. 25, 2021

Related U.S. Application Data
(63) Continuation-in-part of application No. 17/075,486, filed on Oct. 20, 2020.
Application No. 17/075,486 is a continuation-in-part of application No. 16/351,045, filed on Mar. 12, 2019.

(30) Foreign Application Priority Data
Mar. 12, 2018 (IN) 201821008982

(51) Int. Cl. C12P 7/06; C12P 7/10; C12P 7/14

(52) U.S. Cl. CPC C12P 7/065; C12P 7/14; C12P 7/10

(57) ABSTRACT

The present invention relates to a process for continuous production of second-generation ethanol from lignocellulosic biomass via continuous simultaneous saccharification and co-fermentation (SSCF) process, wherein the process includes a first fermentor vessel for selectively fermenting C5 sugars and then Continuous transferring the fermented biomass to a second fermentor vessel for hydrolyzing the fermented biomass and then Continuous transferring the hydrolysate to a third fermentor vessel for selectively fermenting C6 sugars to obtain ethanol. Overall, the ethanol yield achieved was up to 70% for both C5 and C6 sugars from pretreated biomass; and C5 utilization exceeded 95% after SSCF.

### CROSS-REFERENCE TO RELATED APPLICATIONS

The present patent application is a patent of addition of the main Indian Patent Application No. 201821008982 of Filing date Mar. 12, 2018, and Publication date Jan. 3, 2020. The present application comprises an improvement in or a modification of the invention claimed in the specification of the main patent applied for in the Indian Patent Application No. 201821008982.

### FIELD OF THE INVENTION

The present invention relates to a process for continuous production of second-generation ethanol from lignocellulosic biomass in simultaneous saccharification and co-fermentation (SSCF) process. More particularly, the process achieves overall ethanol yield up to 70% for both C5 and C6 sugars from pretreated biomass. C5 utilization exceeds 95% after SSCF. This involves using three separate vessels/fermentors for the fermentation process based on the temperature changes required.

### BACKGROUND OF THE INVENTION

Simultaneous Saccharification and fermentation/co-fermentation (SSF/SSCF) removes sugar inhibition on enzymatic hydrolysis thus increasing the hydrolysis sugar yield and reducing contamination risk. Moreover, SSF/SSCF reduces the overall reaction time and reactor volume (Kristensen et al., 2009). SSF/SSCF sacrifices the optimal conditions for both enzymatic hydrolysis and fermentation. Typically, in enzymatic hydrolysis and fermentation in SSF system the temperature is kept at 37-42° C. as a compromise for better enzymatic hydrolysis and fermentation (Dien et al., 2003b). In addition, SSF/SSCF introduces a new inhibitor (ethanol) for enzymatic hydrolysis. But the inhibitory effect from ethanol is much lower compared to cellobiose or glucose (Taherzadeh & Karimi, 2007).

Continuous approach of SSCF is more economical and practical at demo and production level because it makes the process more economical and less labor-intensive approach. There is a report regarding continuous SSCF by Jin et al. 2013 for ethanol production from Ammonia Fiber Expansion (AFEX™) pretreated corn stover. In this study, the author represented enhanced ethanol production from pretreated corn stover by following pre-hydrolysis by fermentation. In the approach of continuous SSCF, pre-hydrolysis is followed for 24 hours and the then the fermentation is followed in three conjugative fermentors arranged in a bioreactor train fashion. The flow rate in the reactors is similar.

Furthermore, the main Indian Patent Application No. 201821008982 disclosed a batch SSCF process, in which fermentation time reduced significantly with application of low dose of cellulase enzyme in comparison to the conventional SSCF process. However, the present invention discloses a process in a continuous mode which has several advantages over the main Indian Patent Application No. 201821008982 as described below.
Continuous SSCF process reduces operational area reactor vessel in demo or production scale.
Initial viscosity of fermentation process reduces significantly (less than 90%) which makes the process more energy viable.
Dosage of yeast to the fermentor vessel (C5 and C6) will be reduced because yeast will get sufficient time to double its cell biomass during the fermentation process
Desired temperature required for the process will be maintained separately which also makes the process more energy saving.
Reduced chances of contamination as sugar concentration is low, ethanol titer and yeast concentration is higher in comparison to batch process.

Accordingly, the present invention provides a process which overcomes the aforesaid drawback of the prior arts. In the present invention, overall ethanol yield was achieved upto 70% for both C5 and C6 sugars from pretreated biomass. C5 utilization exceeded 95% after SSCF. In the current practice the C5 and C6 sugars were targeted for fermentation in a sequence manner to achieve higher ethanol titer at short time of fermentation and low dose of enzyme.

### SUMMARY OF THE INVENTION

Present invention relates to a process for continuous production of second-generation ethanol from a lignocellulosic biomass, wherein the process includes a first fermentor vessel for fermenting mainly C5 sugars and continuous transferring the fermented biomass to a second fermentor vessel for hydrolyzing the fermented biomass and then continuous transferring the hydrolysate to a third fermentor vessel for selectively fermenting C6 sugars to obtain ethanol. The C5 and C6 sugars are targeted for fermentation in a sequential manner to achieve higher ethanol titer at short time of fermentation (56 hours) and low dose of enzyme. Further, in the present invention of continuous SSCF process, overall ethanol yield up to 70% was achieved for both C5 and C6 sugars from pretreated biomass; and C5 utilization exceeded 95% after SSCF. Therefore, the C5 fermentation, hydrolysis and C6 fermentation is performed in three separate vessels at the required temperature. The present invention ultimately reduces vessel numbers, reduction of continuous yeast dosing to fermentation vessels, low concentration of the xylose maintained in both fermentation vessels throughout the process which reduces the C5 fermentation time and initial higher viscosity problem in batch process is reduced up to 90%, which ultimately saves overall energy input for stirring and no additional filling or emptying time in steady state.

Accordingly, present invention provides a process for continuous production of a second-generation ethanol from a lignocellulosic biomass comprising;
(i) adding slurry of a pre-treated lignocellulosic biomass comprising C5 and C6 sugars in a first fermenting vessel of a fermentor system for a first fermentation process;
(ii) Fermenting mainly C5 sugars by incubating the pretreated lignocellulosic biomass with a cellulase enzyme, a co-fermenting microorganism and a nutrient to obtain ethanol. After 16-20 h, process made continuous by adding pretreated slurry to maintain HRT 16-20 h;
(iii) Continuous transferring fermented biomass of the first fermenting vessel to a second fermenting vessel of the fermentor system for conducting a hydrolysis reaction at 48-55° C. for retention time of 28-30 hours;
(iv) Continuous transferring hydrolysate of the second fermenting vessel to a third fermenting vessel of the fermentor system for a second fermentation process for retention time of 08-12 hours;
(v) fermenting mainly C6 sugars to obtain ethanol.

In one of the features of the present invention, the C5 sugar is selected from xylose and C6 sugar is selected from glucose.

In another feature of the present invention, the concentration of the cellulase enzyme in a range of 1.8-2.5 FPU/TS is employed for the fermentation process.

In yet another feature of the present invention, the fermentation of C5 sugar is carried out at a temperature in a range of 33-35° C. for 16-20 hours.

In still another feature of the present invention, the fermentation of C6 sugar is carried out at a temperature in a range of 35-37° C. for 8-10 hours.

In yet another feature of the present invention, the pre-treated lignocellulosic biomass slurry is added in the first fermenting vessel of the fermentor system of step (i) without any detoxification. In another feature of the present invention, the pH of the slurry of step (i) to 5-5.5 is adjusted with a pH adjuster. The pH adjuster is selected from aqueous ammonium solution, NaOH, KOH, CaCO₃, or a substance which is alkaline in nature and increases pH.

In still another feature of the present invention, the nutrient is MgSO₄ or any other magnesium salt. Nitrogen source such as urea, ammonium sulfate etc is required in case pH adjuster is other than aqueous ammonia.

In still another feature of the present invention, the cellulase enzyme is from fungal or bacterial origin, composed of cellobiohydrolase (I &II), endo-glucanase and β-glucosidase along with other accessory enzyme, wherein the other accessory enzyme is selected from xylanase, β-xyloxidase, arabinofuranosidase, and pectinse or any other enzyme which hydolyyze glucan and/or xylan.

In yet another feature of the present invention, the co-fermenting (C6 and C5 sugar) microorganism is selected from Saccharomyces cerevisiae, or any ethanogenic co-fermenting microorganism such as Pichia sp., Candida sp., Zymomonas mobilis and E. coli.

In still another feature of the present invention, the lignocellulosic biomass is selected from straw, wheat straw, rice straw, sugarcane bagasse, cotton stalk, barley stalk, bamboo or any agriculture residues which contain cellulose or hemicellulose or both.

In one of the features, the present invention provides a process for continuous production of a second-generation ethanol from a lignocellulosic biomass comprising:
(i) adding a slurry of pre-treated lignocellulosic biomass comprising C5 and C6 sugars with 15-20 weight % total solids (TS) and without any detoxification in a first fermenting vessel of a fermenter system for a first fermentation process;
(ii) adjusting pH of the slurry of step (i) to 5-5.5 with aqueous ammonium solution to obtain a pH adjusted slurry;
(iii) fortifying the pH adjusted slurry with MgSO₄ in amount of 03-05 g/l, along with a cellulase enzyme and a co-fermenting microorganism;
(iv) adding water to the slurry of step (iii) to maintain 15-20 weight % TS in the slurry;
(v) incubating the slurry of step (iv) at 33-35° C. for 16-20 hours for a selectively fermenting mainly C5 sugars to obtain ethanol;
(vi) Continuous transferring fermented biomass of the first fermenting vessel to a second fermenting vessel of the fermentor system for conducting a hydrolysis reaction at 48-55° C. for a period of 28-30 hours;
(vii) Continuous transferring hydrolysate of the second fermenting vessel to a third fermenting vessel of the fermentor system for a second fermentation process for at 35-37° C. for 8-10 hours;
(viii) Fermenting mainly C6 sugars to obtain ethanol.

In one of the preferred features, the present invention provides a system for continuous production of a second-generation ethanol from a lignocellulosic biomass, said system comprising: a first fermentor vessel with size of 16000 M³ and hydraulic reaction time (HRT) of 16 hours and dilution rate maintained at 0.0625 h⁻¹; a second fermentor vessel with size of 30000 M³ and HRT of 30 hours and dilution rate maintained at 0.033 h⁻¹; and a third fermentor vessel with size of 10000 M³ and HRT of 10 hours; wherein three fermentor vessels are arranged in a sequential manner; and wherein in-out flow rate to all the fermentor vessels is maintained at a constant rate of 1000M³ to achieve steady state.

### BRIEF DESCRIPTION OF DRAWINGS

FIG. 1 illustrates schematic presentation of the batch and continuous SSCF fermentation process;

FIG. 2 illustrates continuous SSCF process for ethanol production;

FIG. 3 illustrates results of the ethanol produced in continuous fermentation process wherein Fermentor 1 (F1): Xylose (C5) fermentation, Fermentor 2 (F2): Enzymatic hydrolysis and Fermentor 3 (F3): Glucose (C6) fermentation;

### DETAILED DESCRIPTION OF THE INVENTION

While the invention is susceptible to various modifications and alternative forms, specific embodiment thereof will be described in detail below. It should be understood, however that it is not intended to limit the invention to the particular forms disclosed, but on the contrary, the invention is to cover all modifications, equivalents, and alternative falling within the scope of the invention as defined by the appended claims.

### Definition

For the purposes of this invention, the following terms will have the meaning as specified therein: “Pre-treated biomass” or “Pretreatment of biomass” used herein clears away physical and chemical barriers that make native biomass recalcitrant and exposes cellulose for better enzymatic hydrolysis. In most of the pretreatment, chemical (acid or alkali) and physical (high temperature or pressure) parameters are used individually or in mixed manner to remove barriers for enzymatic hydrolysis and improve the enzymatic digestibility.

“Detoxification” used herein is the process where the inhibitors (toxic compound such hydroxymethyl furfural, furfural, acetic acids, formic acids, etc.) produced during the pretreatment process are removed or neutralized from pre-treated biomass by chemical, physical, or biological process.

“Cellulase enzyme” used herein is a mixed form of enzyme which is mostly composed of cellobiohydrolase (I &II), endo-hydrolase and β-glucosidase. This enzyme was mostly produced from fungal sources. Cellulase breaks down the cellulose molecule into monosaccharide and shorter polysaccharides or oligosaccharides. In the present invention the cellulase enzyme is selected from commercially available cellulase enzymes which are suitable for the purposes.

“C5 sugars” used herein represents xylose.

“C5 fermentation” used herein is xylose fermentation into ethanol.

“C6 sugar” used herein represents glucose.

“C6 fermentation” used herein is glucose fermentation into ethanol.

“Nutrient” used herein is MgSO₄. In the salt MgSO₄ used in fermentation where, Mg⁺² acts as an essential enzyme cofactor and acts as key structural component of most biological pathways. During fermentation Mg⁺² plays a major role for proper functioning of fermenting enzymes in yeast.

Simultaneous saccharification and co-fermentation (SSCF) is a promising strategy for obtaining high ethanol yield. This process operates in a single fermentor vessel where the required temperature keeps changing during the fermentation practice. But shifting of temperature from higher to lower in a batch process makes the process more energy intensive at higher operation level (demo or production scale). Also, large scale plant reactors would require transferring volume from one vessel to another vessel of different temperatures, which would require additional emptying and filling time. This would result in higher capital and operational cost to the process. To overcome this issue, in the present invention, the temperature changes (33° C. for C5 fermentation, 50° C. for hydrolysis and 35° C. for C6 fermentation) for fermentation process are performed in three separate fermentor vessels sequentially at their desired temperatures. This makes the process easier, however, at large scale operation two bioreactor train system (i.e., three vessels in each train in a parallel manner, in total six vessels required) arrangements are required to make the process continuous. Continuous fermentation process reduces vessel number, reduction of continuous yeast dosing to fermentation vessel, low concentration of xylose maintained in both fermentation vessels throughout the process which reduces the C5 fermentation time and initial higher viscosity problem in batch process is reduced up to 90%, which ultimately saves overall energy input for stirring. In the process of the present invention, the C5 and C6 sugars targeted for fermentation in sequence manner to achieve higher ethanol titer at short time of fermentation and low dose of enzyme. An overall ethanol yield up to 70% was achieved for both C5 and C6 sugars from pretreated biomass. C5 utilization also exceeded 95% after SSCF.

The present invention discloses a process for continuous production of second-generation ethanol from lignocellulosic biomass in SSCF process using three separate vessels/fermentors for the fermentation process based on the temperature changes required.

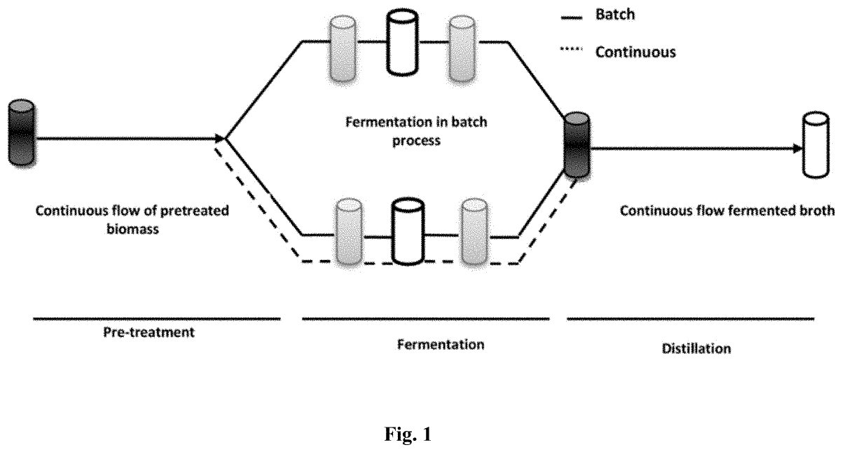

A continuous SSCF process as disclosed in the present invention when compared to a batch SSCF process (see FIG. 1). In this continuous SSCF approach three bioreactors are attached conjugative as described in FIG. 1. The batch SSCF process and continuous SSCF process are represented in yellow and red colored line, respectively. In (pretreated biomass) and out (fermented broth) flow (presented in arrow line) of the process are in continuous mode. When all vessels in the described continuous SSCF process reached to the designated volume then the flow rate to the entire reactor maintained in a constant rate. The improvements of the continuous SSCF over batch SSCF are given below in Table 1.

TABLE 1
Improvements of the Continuous SSCF over Batch SSCF

| | Continuous SSCF | Batch SSCF | Remark |
|---|---|---|---|
| Reactor Volume | 16000 + 30000 + 10000 = | (12000 + 12000 + 12000) * 2 = | Less working volume |
| | 56000 M³ | 72000 M³ | reduces final cost to |
| | | | the process |
| Viscosity | >1000 cP | Very high (Initial 1 lakh | Low viscosity |
| | | cP) | improves |
| | | | fermentation mass |
| | | | flow and electric |
| | | | saving |
| Yeast Dozing | Not required after | Every batch requires | One time yeast |
| | pitching | pitching | pitching at starting of |
| | | | process. This reduces |
| | | | yeast cost by |
| | | | approximately one |
| | | | rupee/liter of ethanol |
| Temperature | Constant in reactors | Variable (up and down) | Constant temperature |
| | | | in the fermentor |
| | | | makes the process |
| | | | easier and economical |
| | | | for operation. |

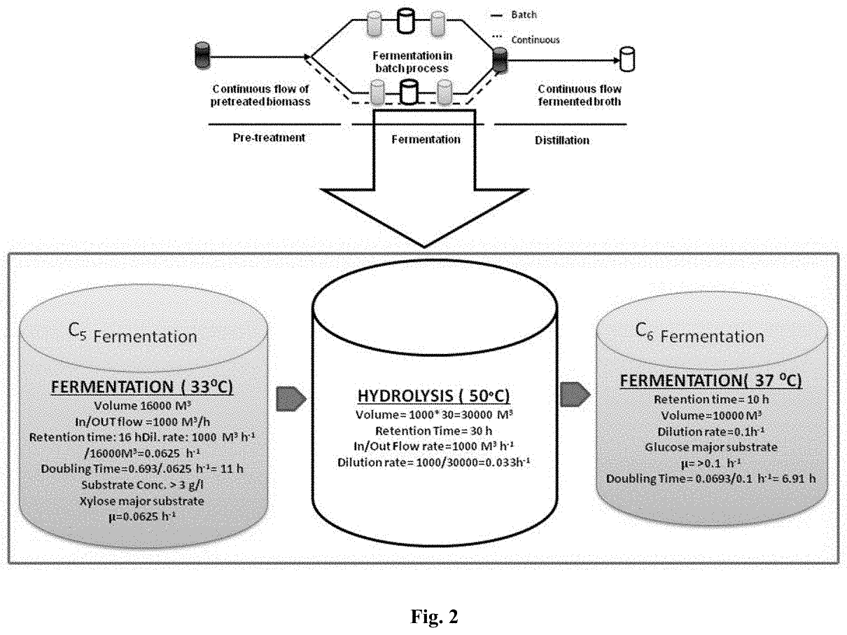

A process for continuous production of a second-generation ethanol from a lignocellulosic biomass (see FIG. 2) comprising:
(i) adding a slurry of pre-treated lignocellulosic biomass comprising C5 and C6 sugars with 20-22 weight % total solids (TS) and without any detoxification in a first fermenting vessel of a fermenter system for a first fermentation process;
(ii) adjusting pH of the slurry of step (i) to 5-5.5 with aqueous ammonium solution to obtain a pH adjusted slurry;
(iii) Fortifying the pH adjusted slurry with MgSO₄ in amount of 03 g/l, along with a cellulase enzyme and a co-fermenting microorganism;
(iv) adding water to the slurry of step (iii) to maintain 15-20 weight % TS in the slurry;
(v) incubating the slurry of step (iv) at 33-35° C. for 16-20 hours for a fermenting mainly C5 sugars to obtain ethanol;
(vi) Continuous transferring fermented biomass of the first fermenting vessel to a second fermenting vessel of the fermentor system for conducting a hydrolysis reaction at 48-55° C. for a period of 28-30 hours;
(vii) Continuous transferring hydrolysate of the second fermenting vessel to a third fermenting vessel of the fermentor system for a second fermentation process for at 35-37° C. for 8-10 hours;
(viii) Fermenting mainly C6 sugars to obtain ethanol.

In accordance with the present invention, steady state ethanol production achieved using dilute acid pretreated rice straw. In this process, three reactors of different volume are in series with constant flow of slurry. However, different HRT maintained by different volume size of reactor. First reactor utilized for preferential xylose fermentation at 33° C. followed by hydrolysis in second reactor at 50° C. Thereafter, slurry moved to third reactor for mainly glucose fermentation at 37° C. This resulted into several advantages which are tabulated in Table-1.

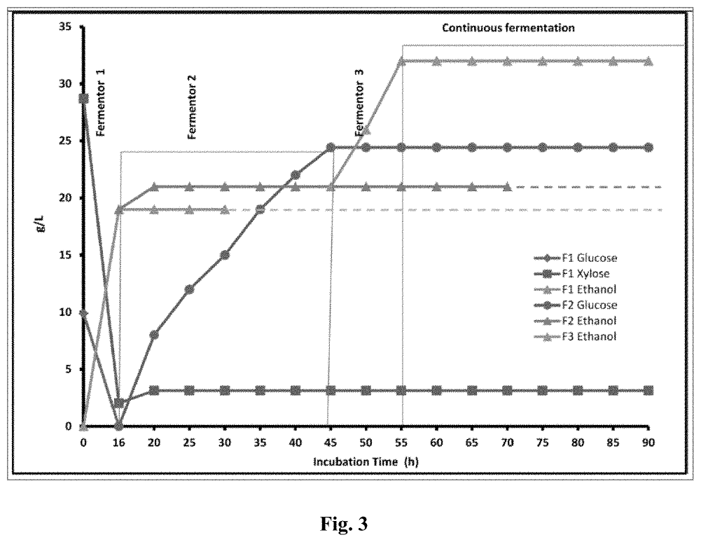

In another feature of the present invention, when all vessels in the described continuous SSCF process reached to the designated volume then the flow rate to the entire reactor maintained in a constant rate. All the flow and the dilution in the fermentor vessel are maintained as described in the FIG. 2. In FIG. 2, first fermentor/vessel represent for pentose sugar fermentation, middle fermentor/vessel represent for enzymatic hydrolysis and third fermentor/vessel for hexose sugar utilization. Respective volume, flow and dilution rates are mentioned. At final fermentation 32 g/L ethanol concentration produced in third fermentor and continued in steady state. At the same time in other fermentor (First and second) sugar release and ethanol concentration are in steady state. This steady state achieved after 56 hours of the fermentation (see FIG. 3). In FIG. 3, Fermentor 1 (F1): Xylose (C5) fermentation, Fermentor 2 (F2): Enzymatic hydrolysis and Fermentor 3 (F3): Glucose (C6) fermentation is disclosed. This fermentation process followed the continuous process after 55 hours of fermentation and in continuous fermentation achieved after when the steady state is achieved in all three fermenters.

### Example 1

Process for Continuous Production of Second-Generation Ethanol from a Lignocellulosic Biomass:

Pretreated biomass (slurry, TS approximately 20-22%) without any detoxification is introduced directly to the first fermentor vessel of the fermentor system. The pH of the slurry was adjusted to 5-5.5 with aqueous ammonium solution (25% initial concentration). The pH adjusted slurry was fortified with MgSO₄ (0.5%), cellulase enzyme (in-house enzyme/Ctec, 2.3 FPU/TS) and co-fermenting ethanologenic yeast Saccharomyces cerevisiae (1 g dry cell biomass/100 gTS, xylose utilizing genetically modified yeast). Required amount of water was added to the process to maintain the final biomass concentration to 15%. The whole process was incubated at 33° C. for 16 hours for the xylose fermentation. The fermented broth is then transferred to second fermentor vessel of the fermentor system and is allowed for hydrolysis at 50° C. for 30 hours. The volume of the reactor is maintained at 1.87 times higher as compared to pentose sugar utilizing fermentor vessel for giving hydraulic reactor time of 30 hours. After the hydrolysis, the hydrolysate is transferred to third fermentor vessel of a fermenting system and for the hexose sugar fermenting vessel for 10 hours. All reactors have 1000 M³ flow. When all vessels in the described continuous SSCF process reached to the designated volume then the flow rate to the entire reactor was maintained in a constant rate. At final fermentation, 32 g/L ethanol concentration was produced in third fermentor vessel and continued in steady state. At the same time in other fermentor (First and second) vessels, sugar release and ethanol concentration are in steady state. This steady state was achieved after 56 hours of the fermentation. The results of this experiment are represented by FIG. 2.

### Example 2

Process for Batch Production of Second-Generation Ethanol from a Lignocellulosic Biomass (Main Indian Patent Application No. 201821008982)

The pH of the pretreated slurry was adjusted to 5.5 with aqueous ammonium solution (25% initial concentration). The pH adjusted slurry was fortified with 3 g/l MgSO₄, cellulase enzyme (Commercial enzyme, 3.3 FPU/TS) and co-fermenting Saccharomyces cerevisiae (1 g dry cell biomass/litre, xylose and glucose utilizing yeast). Required amount of water was added to the process to adjust the final biomass concentration to 20%. The whole process was incubated at 30° C. for 16 h for the fermentation with 200 rpm. When the free xylose concentration in the slurry comes near to 6-7 g/1, the temperature of the process was increased to 33° C. and 35° C., incubated for 2 h in each temperature for better hydrolysis and fermentation. After that temperature increased to 48° C. This step mainly required for rapid releases of glucose sugar from cellulose which converted simultaneously with hydrolysis to ethanol by yeast biomass. As the temperature was reached at desired target the process was allowed to maintain the required temperature (48° C.) for 23 h for better enzymatic hydrolysis. After this incubation the system was allowed to cool down to temperature 35° C. A second dose of co-fermenting S. cerevisiae (1 g dry cell biomass/liter) was inoculated to the system for the second stage of fermentation. The second fermentation was stopped after 6 h of fermentation. This process took 46 h incubation including fermentation and enzymatic hydrolysis.

In the present invention, the continuous SSCF approach is considered as advantageous over the conventional SSCF and batch SSCF due to several reasons as described in Table-1.

### REFERENCES

1. Krishnan, C., Sousa, L. D., Jin, M. J., Chang, L. P., Dale, B. E., Balan, V. 2010. Alkali-based AFEX Pretreatment for the conversion of sugarcane bagasse and cane leaf residues to ethanol. Biotechnology and Bioengineering, 107(3), 441-450.
2. Dien, B. S., Cotta, M. A., Jeffries, T. W. 2003. Bacteria engineered for fuel ethanol production: current status. Applied Microbiology and Biotechnology, 63(3), 258-266.
3. Taherzadeh, M. J., Karimi, K. 2007. Enzyme-based hydrolysis processes for ethanol from lignocellulosic materials: a review. Bioresources, 2(4), 707-738.
4. Lau, M. W., Dale, B. E. 2009. Cellulosic ethanol production from AFEX-treated corn stover using Saccharomyces cerevisiae 424A (LNH-ST). Proceedings of the National Academy of Sciences of the United States of America, 106(5), 1368-1373.
5. Jin M., Gunawan C., Balan V., Yu X., Dale B. E. 2013. Continuous SSCF of AFEX™ pretreated corn stover for enhanced ethanol productivity using commercial enzymes and Saccharomyces cerevisiae 424A (LNH-ST). Biotechnology and Bioengineering, 110, 5, 1302-1311.

What is claimed is:

1. A process for continuous production of a second-generation ethanol from a lignocellulosic biomass comprising;
(i) adding slurry of a pre-treated lignocellulosic biomass comprising C5 and C6 sugars in a first fermenting vessel of a fermentor system for a first fermentation process;
(ii) Fermenting mainly C5 sugars by incubating the pretreated lignocellulosic biomass with a cellulase enzyme, a co-fermenting microorganism and a nutrient to obtain ethanol;
(iii) Continuous transferring fermented biomass of the first fermenting vessel to a second fermenting vessel of the fermentor system for conducting a hydrolysis reaction at 48-55° C. for a period of 28-30 hour;
(iv) transferring hydrolysate of the second fermenting vessel to a third fermenting vessel of the fermentor system for a second fermentation process for 8-10 hour;
(v) Fermenting mainly C6 sugars to obtain ethanol.

2. The process as claimed in claim 1, wherein the C5 sugar is selected from xylose and C6 sugar is selected from glucose.

3. The process as claimed in claim 1, wherein the concentration of the cellulase enzyme in a range of 1.8-2.5 FPU/TS is employed for the fermentation process.

4. The process as claimed in claim 1, wherein the fermentation of C5 sugar is carried out at a temperature in a range of 33-35° C. for 16-20 hour.

5. The process as claimed in claim 1, wherein the fermentation of C6 sugar is carried out at a temperature in a range of 35-37° C. for 08-10 hour.

6. The process as claimed in claim 1, wherein the pre-treated lignocellulosic biomass slurry is added in the first fermenting vessel of the fermentor system of step (i) without any detoxification.

7. The process as claimed in claim 1, additionally comprising adjusting pH of the slurry of step (i) to 5-5.5 with a pH adjuster.

8. The process as claimed in claim 7, wherein the pH adjuster is selected from aqueous ammonium solution, NaOH, KOH, CaCO₃ or a substance which is alkaline in nature and increases pH.

9. The process as claimed in claim 1, wherein the nutrient is MgSO₄ or any other magnesium salt. Nitrogen source such as urea, ammonium sulfate etc is required in case pH adjuster is other than aqueous ammonia.

10. The process as claimed in claim 1, wherein the cellulase enzyme is from fungal or bacterial origin, composed of cellobiohydrolase (I, II), endo-glucanase and β-glucosidase along with other accessory enzyme, wherein the other accessory enzyme is selected from xylanase, β-xyloxidase, arabinofuranosidase, and pectinse or any other enzyme which hydolyze glucan and/or xylan.

11. The process as claimed in claim 1, wherein the co-fermenting microorganism is selected from Saccharomyces cerevisiae, or any ethanogenic co-fermenting microorganism such as Pichia sp., Candida sp., and E. coli.

12. The process as claimed in claim 1, wherein the lignocellulosic biomass is selected from straw, wheat straw, rice straw, sugarcane bagasse, cotton stalk, barley stalk, bamboo or any agriculture residues which contain cellulose or hemicellulose or both.

13. A process for continuous production of a second-generation ethanol from a lignocellulosic biomass comprising:
(i) adding a slurry of pre-treated lignocellulosic biomass comprising C5 and C6 sugars with 20-22 weight % total solids (TS) and without any detoxification in a first fermenting vessel of a fermenter system for a first fermentation process;
(ii) adjusting pH of the slurry of step (i) to 5-5.5 with aqueous ammonium solution to obtain a pH adjusted slurry;
(iii) fortifying the pH adjusted slurry with MgSO₄ in amount of 03 g/l, along with a cellulase enzyme and a co-fermenting microorganism;
(iv) adding water to the slurry of step (iii) to maintain 15-20 weight % TS in the slurry;
(v) incubating the slurry of step (iv) at 33-35° C. for 16-20 hours for a selectively fermenting mainly C5 sugars to obtain ethanol;
(vi) Continuous transferring fermented biomass of the first fermenting vessel to a second fermenting vessel of the fermentor system for conducting a hydrolysis reaction at 48-55° C. for a period of 28-30 hours;
(vii) Continuous transferring hydrolysate of the second fermenting vessel to a third fermenting vessel of the fermentor system for a second fermentation process for at 35-37° C. for 08-10 hours;
(viii) Fermenting mainly C6 sugars to obtain ethanol.

14. A system for continuous production of a second-generation ethanol from a lignocellulosic biomass, said system comprising:
a first fermentor vessel with size of 16000 M³ and hydraulic reaction time (HRT) of 16 hours and dilution rate maintained at 0.0625 h⁻¹;
a second fermentor vessel with size of 30000 M³ and HRT of 30 hours and dilution rate maintained at 0.033 V; and
a third fermentor vessel with size of 10000 M³ and HRT of 10 hours;
wherein three fermentor vessels are arranged in a sequential manner; and
wherein in-out flow rate to all the fermentor vessels is maintained at a constant rate of 1000M³ to achieve steady state.
